(19) United States
(12) Patent Application Publication
(10) Pub. No.: US 2003/0211519 A1
(43) Pub. Date: Nov. 13, 2003

(54) Uses of an endothelial cell receptor

(76) Inventor: Donald J. Davidson, Gurnee, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/322,853

(22) Filed: Dec. 18, 2002

Related U.S. Application Data
(60) Provisional application No. 60/342,029, filed on Dec. 19, 2001.

(51) Int. Cl. C12Q 1/68; G01N 33/567; G01N 33/53; C07K 14/705; C12P 21/02; C12N 5/06

(52) U.S. Cl. 435/6; 435/7.2; 435/455; 435/325; 530/350

(57) ABSTRACT

The subject invention relates to uses of a receptor referred to as GRP78 and to other endothelial cell receptors which bind to the kringle 5 region of mammalian plasminogen. More specifically, identification of the functional properties of this receptor and other such receptors allows for the development and screening of agents which, for example, mimic K5 (i.e, mimetics) and therefore inhibit angiogenesis.

The present application claims priority to U.S. provisional patent application serial No. 60/342,029, filed on Dec. 19, 2001, hereby incorporated in its entirety by reference.

### BACKGROUND OF THE INVENTION

1. Technical Field

The subject invention relates to uses of a receptor referred to as GRP78 and to other endothelial cell receptors which bind to the kringle 5 region of mammalian plasminogen. More specifically, identification of the functional properties of this receptor and other such receptors allows for the development and screening of agents which, for example, mimic K5 (i.e, mimetics) and therefore inhibit angiogenesis.

2. Background Information

Angiogenesis is the process in the body by which new blood vessels are formed. This process is essential for normal body activities including, for example, reproduction, development and wound repair. Under normal biological conditions, angiogenesis is a highly regulated process. However, many diseases are driven by persistent, unregulated angiogenesis.

Several angiogenesis inhibitors are under development or have been developed for use in treating angiogenic diseases (Gasparini et al., J. Clin. Oncol., 13(3):765-782 (1995). Such inhibitors include, for example, suramin and K5. (For a discussion of the properties of K5, see, e.g., Cao et al., Journal of Biol. Chem. 272:22924-22928 (1997), Ji et al., Biochem. and Biophys. Res. Communs. 247:414-419 (1998) and Lu et al., Biochem. and Biophys. Res. Communs. 258:668-673 (1999).) Thus, by analyzing the receptors to which angiogenic inhibitors bind and, in particular, the binding interaction or relationship itself, one may screen for further angiogenic inhibitors as well as purposefully design these inhibitors.

The present inventors have determined that one receptor to which K5 binds is GRP78. This molecular chaperone is constitutively expressed and expression is often dramatically enhanced under stressful conditions such as glucose deprivation, treatment with Ca2+ ionophores, blockage of glycosylation, oxidative stress and hypoxia (Song et al., Cancer Research 61:8322-8330 (2001)). GRP78, also referred to as the immunoglobulin heavy chain binding protein BIP, also plays a role in protecting tumor cells against cytotoxic T lymphocyte-mediated toxicity and the toxic effects of tumor necrosis factor in vitro (Jamora et al., PNAS 93:7690-7694 (1996); see also Lee, A., TRENDS in Biochemical Sciences, Vol. 26, No. 8, pps. 504-510 (2001)).

In view of the characteristics of the GRP78 protein, as noted above, and the fact that K5 binds to this protein, there is an essential need for other agents, similar to K5, which can bind thereto. Such agents may be used to inhibit angiogenesis as well as other functions of the GRP78 receptor.

All U.S. patents and publications referred to herein are hereby are incorporated in their entirety by reference.

### SUMMARY OF THE INVENTION

The present invention encompasses a method of identifying a composition which inhibits activation of an endothelial cell receptor. The method comprises constructing a vector comprising a nucleotide sequence encoding the endothelial cell receptor and a nucleotide sequence encoding a reporter molecule. The nucleotide sequence encoding the reporter molecule is operably linked to the nucleotide sequence encoding the endothelial cell receptor, introducing the vector into a host cell for a time and under conditions suitable for expression of the endothelial cell receptor, exposing the host cell to a composition which may inhibit activation of the endothelial cell receptor and a substrate specific for the reporter molecule, and measuring the signal generated by reaction of said reporter molecule and said substrate in comparison to that produced by a control host cell, a smaller signal by the host cell into which the modified vector was introduced, indicating that the composition will inhibit activation of the endothelial cell receptor. The receptor may be, for example, GRP78. An example of the composition is K5.

A further embodiment of the present invention encompasses a method of identifying a composition which inhibits expression of an endothelial cell receptor comprising the steps of adding an antibody selected from the group consisting of a monoclonal antibody and a polyclonal antibody produced against the endothelial cell receptor to a solid phase, adding known concentrations of the endothelial cell receptor exposed to the test composition, to the solid phase, in order to form a first complex between the antibody and the known concentrations of the endothelial cell receptor, adding a second antibody to the first complex, selected from the group consisting of a monoclonal antibody and a polyclonal antibody produced against the endothelial cell receptor for a time and under conditions sufficient for formation of a second complex between the first complex and the second antibody, contacting the second complex with an indicator reagent which comprises a signal-generating compound attached to an antibody against the antibody of the second complex, for a time and under conditions sufficient for formation of a third complex, and detecting the presence of a measurable signal, absence of the signal indicating the composition inhibits expression of the endothelial cell receptor and presence of the signal indicating the composition does not inhibit expression of the endothelial cell receptor. The endothelial cell receptor is, for example, GRP78. The composition which inhibits expression of the receptor may be, for example, K5.

A further embodiment of the present invention includes a method of identifying a composition which binds to the GRP78 receptor comprising the steps of exposing the receptor to said composition for a time and under conditions sufficient for formation of a complex and determining presence or absence of said complex, presence of the complex indicating a composition which binds to the receptor. The composition may be attached to an indicator molecule capable of generating a detectable signal. The composition which binds to the GRP78 receptor may be, for example, K5 or a functional equivalent thereof.

Additionally, the present invention includes a method of preventing or treating angiogenesis in a patient in need of such prevention or treatment comprising the step of administering an amount of a composition which binds to at least one endothelial cell receptor sufficient to effect the prevention or treatment. The endothelial cell receptor may be, for example, GRP78, and the composition may be, for example, K5.

### BRIEF DESCRIPTION OF THE DRAWINGS

FIG. 1 illustrates inhibition of I¹²⁵dog-K5 binding to EAHY cells by a polyclonal antibody to GRP78.

FIG. 2 illustrates inhibition of I¹²⁵dog-K5 binding to EAHY cells by various polyclonal antibodies.

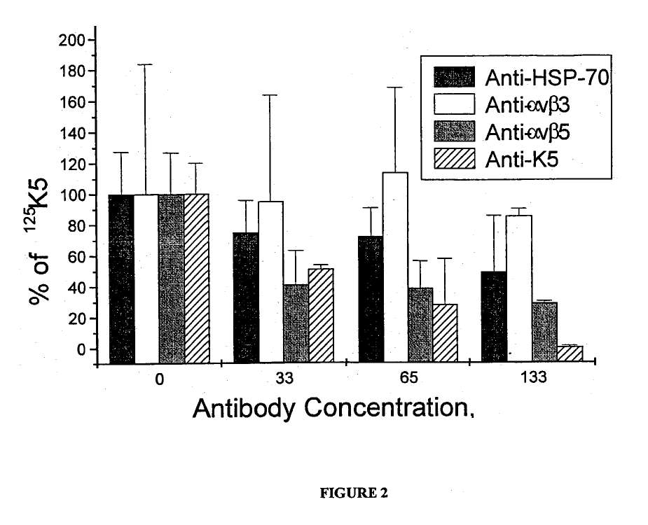

FIG. 3 illustrates inhibition of rK5 activity on migration of HMVEC cells with [symbol] -GRP78.

FIG. 4 the percent inhibition of proliferation when HUAVEC cells were incubated with rK5 and various concentrations of GRP78 antibody.

FIG. 5 represents avidin-HRP blots of biotinylated cell surface proteins isolated by affinity purification with agarose-K5.

FIG. 6 illustrates GRP78 in EAHY cells starved and then fed at various intervals.

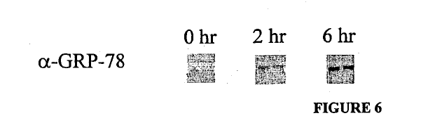

FIG. 7 a determination of the amount of GRP78 present in HMVEC cells starved, exposed to VEGF and stained with a goat polyclonal GRP78 antibody and an anti-goat HRP antibody.

FIG. 8 illustrates the direct binding of recombinant kringle 5 (rK5) with the GRP78 receptor.

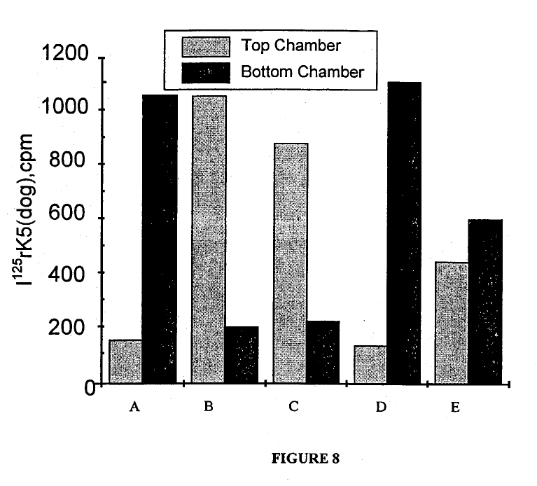

### DETAILED DESCRIPTION OF THE INVENTION

As noted above, the present inventors have discovered that K5 and, in particular, the active site (PRKLYDY) thereof, binds to the endothelial cellular receptor GRP78. Based upon this finding, this protein may be utilized for many purposes. For example, the protein may be used to screen for and identify analogs or mimetics of K5 which bind to the protein also and should therefore be functional equivalents of K5. Such analogs or mimetics may inhibit or suppress angiogenesis in a patient. One may also screen for antagonists and allosteric modulators of the receptor, thereby also reducing or preventing angiogenesis in the patient. One may also screen for agonists of the receptor. (For purposes of the present invention, a “functional equivalent” is defined as a compound or entity which behaves in the same manner, in terms of binding, as the entity to which it is being compared.) Additionally, one may use the receptor to identify compositions that inhibit expression of the receptor. Moreover, the protein may be used in order to further comprehend the binding properties of K5 to a receptor on the cell surface.

Once a useful pharmaceutical composition is identified, it may comprise a therapeutically effective amount of the inhibitor or modulator and an appropriate physiologically acceptable carrier (e.g., water, buffered water or saline). The dosage, form (e.g., suspension, tablet, capsule, etc.), and route of administration of the pharmaceutical composition (e.g., oral, topical, intravenous, subcutaneous, etc.) may be readily determined by a medical practitioner and may depend upon such factors as, for example, the patient's age, weight, immune status, and overall health.

Another embodiment of the present invention encompasses a method of assaying test samples (e.g., biological fluids) for the presence or absence of the GRP78 receptor. Thus, for example, a patient having a malignancy may be tested for presence of the receptor based upon the binding assays described herein.

The drug screening assays referred to above will now be described in detail. For example, in one method, a vector is created comprising an isolated DNA sequence encoding the GPR78 receptor. This sequence may be attached to, for example, a nucleotide sequence encoding a reporter molecule (e.g., an enzyme such beta-galactosidase) or entity capable of interacting with a substrate, thereby emitting or generating a measurable signal. The vector may be, for example, a plasmid, a bacteriophage or a cosmid. The vector is then introduced into host cells under time and conditions suitable for expression of the receptor. (The host cells may be prokaryotic or eukaryotic cells.) The host cells are then exposed to the test composition thought to, for example, inhibit activation of the receptor. The cells are also exposed to the relevant substrate. One then measures the quantity of signals emitted from the reporter molecule-substrate reaction. If the amount of signals produced by the host cells, exposed to the composition in question, is lower than that produced by control cells (i.e., cells which have not been exposed to the composition), then the composition has inhibited the activity of the receptor. If the amount of signal produced by the treated cells is equal to that produced by the control cells, the composition has not inhibited the activity of the receptor. (See, e.g., U.S. Pat. No. 5,912,122, U.S. Pat. No. 5,912,120 and U.S. Pat. No. 5,919,450.)

Additionally, the present invention covers an Affinity-Selection method, using purified receptor in a filtration assay, to identify compositions that bind to the receptor to prevent the receptor from binding to other agents, interacting with agents, etc., thus preventing the receptor from functioning as it would normally in vivo. Briefly, purified receptor is mixed with several test compounds. The mixture is passed through a filter which only allows certain molecular weight molecules to pass through. Compositions that bind to the receptor will be retained by the filter. The unbound compounds are not retained and can be separated from the bound compositions. The structures of the compositions which bind to the receptor are determined, for example, by Mass Spectrometry.

Furthermore, the present invention also encompasses a receptor binding method using radiolabeled receptor to bind to cells or membranes prepared from tissues or cells containing GRP78 receptors. In this manner, one may identify compositions that block GRP78 from binding to agents to which it would normally bind, thus preventing the receptor from functioning. In particular, the purified recombinant receptor protein from, for example, mammalian cells is radiolabeled ([¹²⁵I], [³H], [¹⁴C], etc.). The radiolabeled receptor is then incubated with cells or membranes prepared from tissues or cells which contain the GRP78 receptors in the presence or absence of the test composition. Radiolabeled cells and membranes are then separated from non-radiolabeled cells and membranes by separation methods such as, for example, filtration and centrifugation. The amount of receptor binding to cells or membranes is determined by counting radioactivity. A decrease in radioactivity in the presence of a test composition indicates that the composition inhibits receptor binding, and thus is useful in inhibiting receptor function.

The present invention also covers two methods, using which identify compositions that inhibit the synthesis and expression of the receptor. In the sandwich method, a mammalian monoclonal and/or polyclonal antibody (e.g., rabbit or mouse) against the mature form of the receptor is coated on a solid surface (e.g., the Immulon-4 plate (Dynatech Laboratories Inc., Chantilly, Va.)). The surface will be blotted by a known blotting agent, for example, Bovine Serum Albumin (BSA), and washed. Samples or known concentrations of purified GRP78 are added to the surface (e.g., plate). After the receptor binds to the antibody or antibodies, the surface will be washed, and then incubated with a mammalian monoclonal and/or polyclonal antibody (e.g., goat, rabbit or mouse) raised against the receptor. The binding of the second anti-receptor antibody will be detected by use of an indicator reagent which comprises an antibody conjugated with a signal-generating compound, for example, an enzyme. A substrate for the enzyme is also added if an enzyme is utilized. For example, horseradish peroxidase (HRP) and its substrate O-Phenylenediamine hydrochloride (OPD) may be utilized. In particular, the enzyme-substrate reaction generates a detectable signal or change, for example, color, which may be read, for example, in a Microplate Reader. Examples of signal generating compounds, other than an enzyme which may be utilized include, for example, a luminescent compound, a radioactive element, a visual label and a chemiluminescent compound. Known concentrations of the receptor are used to generate a standard curve. The concentration of receptor in the unknown samples can be determined using the standard curve. The test agents that decrease the receptor concentration in supernatants are potentially useful for inhibition of receptor synthesis on the endothelial cell.

In the competitive method, a fixed amount of the receptor is coated on a solid surface, for example, the Immulon-4 plate. The plate will be blotted by, for example, BSA or another known blotting agent, and washed. Samples are added to the plate along with a mammalian monoclonal and/or polyclonal antibody (e.g., goat, rabbit or mouse) against the receptor. The plate is washed, and then incubated with an indicator reagent comprising an antibody conjugated with a signal-generating compound, for example, an enzyme (or the entities described above). If an enzyme is used, a substrate for the enzyme is also provided. The enzyme may be, for example, horseradish peroxidase (HRP) The substrate may therefore be 0Phenylenediamine hydrochloride (OPD)). Again, the enzyme-substrate reaction generates a detectable change or signal, for example, color, which can be read in, for example, a microplate reader. Known concentrations of purified receptor may be used to generate a standard curve. The concentration of receptor in the unknown samples can be determined using the standard curve. The test agents which decrease the receptor concentration in supernatants are potentially useful for inhibition of receptor synthesis by the cell. Known concentrations of the receptor, or receptor in the sample, compete with receptor protein coated on the plate in binding to receptor antibodies. When more receptor is present in the sample, a smaller signal is generated. If a test agent is able to block receptor, the amount of receptor in that particular sample will be less than in the control, and the signal in that sample will be more than in the control.

The present invention may be illustrated by the use of the following non-limiting examples:

### EXAMPLE I

### Identification of an Endothelial Cell K5 Receptor

The normal function of GRP78 is to chaperone and help fold proteins in the endoplasmic reticulum. Under stressed conditions, unfolded or improperly folded proteins are chaperoned by GRP78 to proteosomes for degradation. Under hypoxic stressed conditions, GRP78 and a close relative to GRP96, HSP90, are found on cell surfaces. Published reports of over expression, antisense, and ribozyme approaches in tissue culture systems suggest that GRP78 can protect cells against cell death. In a variety of cancer cell lines, solid tumors and human biopsies, the level of GRP78 is elevated, correlating with malignancy. In addition, induction of GRP78 has been shown to protect cancer cells from immune surveillance and apoptosis, whereas suppressing the stress-mediated induction of GRP78 enhanced apoptosis, inhibited tumor growth and increased the cytotoxicity of chronic hypoxic cells.

To determine if GRP78 is a cell surface receptor for K5, a goat polyclonal antibody to GRP78 was used to compete with K5's binding to EAHY cells. EAHY cells (20,000 per well) were let adhere to 96 well plates; the cells were then incubated with α-GRP78 and ¹²⁵dog-K5 for 1 hour at 4 C. Media was removed and the cells were washed 5× with cold PBS. Cells were lysed and bound I¹²⁵dog-K5 counted. Assays were run with eight replicates each.

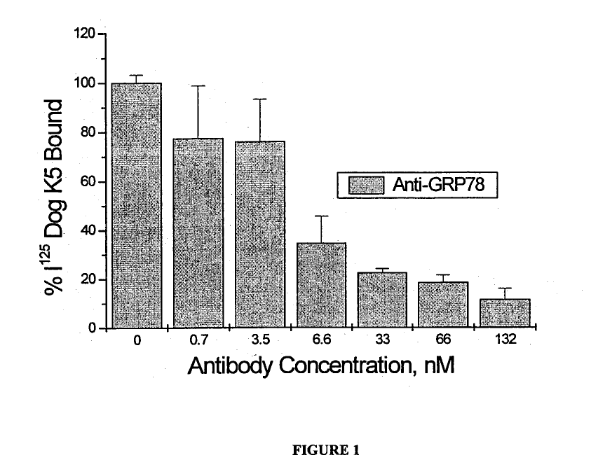

The polyclonal antibody to GRP78 inhibited the binding of 5 nM I¹²⁵K5 (dog) in a dose dependent manner with and IC50 about 6 nM (FIG. 1). As a comparison, EAHY cells (20,000 per well) were let adhere to 96 well plates. The cells were then incubated with the various antibodies and I¹²⁵dog-K5 for 1 hour at 4 C. The media was removed and the cells were washed 5× with cold PBS. Cells were lysed and bound I¹²⁵dog-K5 counted. Assays were run with eight replicates each.

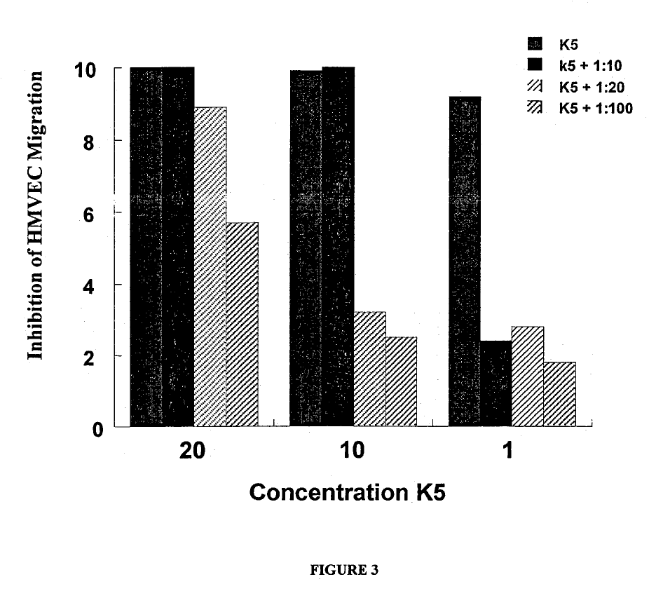

A monoclonal antibody raised against K5 also inhibited I¹²⁵K5 (dog)'s binding to EAHY cells with an IC50 around 15-20 nM, and the panel of various goat polyclonal antibodies weakly inhibited K5's binding to EAHY cells (FIG. 3).

### EXAMPLE II Inhibition of Recombinant K5 Activity on Migration of HMVEC Cells with [symbol] -GRP78

Since the antibody to GRP78 could inhibit K5's binding to endothelial cells, it should also inhibit K5's activity on endothelial cell migration and proliferation.

In particular, MVEC cells were labeled with Casein-AM. The cells were loaded on to the top chamber of a 96 well migration plate. The bottom wells were preloaded with media containing VEGF (10 ng/ml), rK5 (100 nM) and various concentrations of α-GRP78. The plates were incubated at ³⁷C for 4 hours. Membranes were removed and the underside was counted with a fluorometer for cell migration. Assays were run in triplicate. The data obtained is shown in FIG. 3.

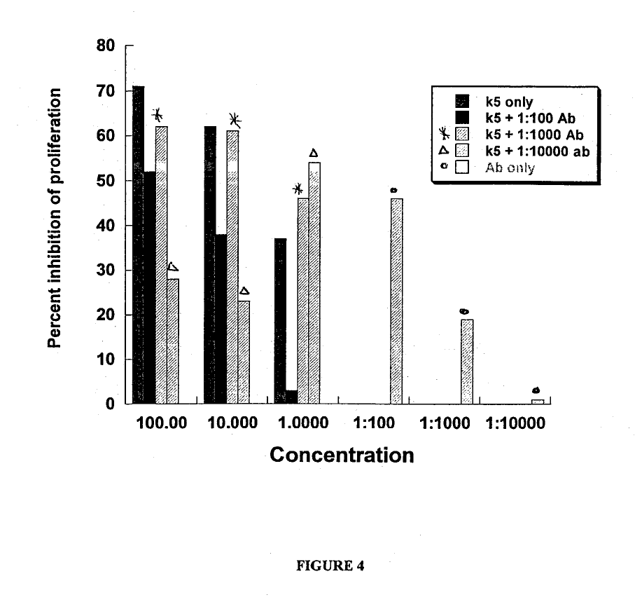

Additionally, HUAVEC cells were incubated with rK5 and various concentrations of GRP78 antibody. The amount of labeled thymidine incorporated was determined after 24 hours and was used to calculate percent inhibition of proliferation compared to untreated cells. The green line displays proliferation inhibition of cells with α-GRP78 alone. As can been seen in FIG. 4, the α-GRP78 at higher concentrations does inhibit cell proliferation, however at lower concentrations (1:10000) this inhibition is not observed. At a 1:10,000 dilution of α-GRP78, the inhibition of K5 activity on endothelial cell proliferation was dose dependent.

In view of the above, in HMVEC migration (FIG. 3) and HUAEC proliferation assays (FIG. 4), anti-GRP78 inhibited rK5's activity in a dose dependent manner.

### EXAMPLE III

### Avidin-HRP Blots of Biotinylated Cell Surface Proteins Isolated by Affinity Purification with AGAROSE-K5

To determine if GRP78 is found on the cell surface of stimulated cells, surface proteins on EAHY cells were labeled with biotin. The cells were then lysed and affinity purification of S-tag-K5 binding proteins was performed.

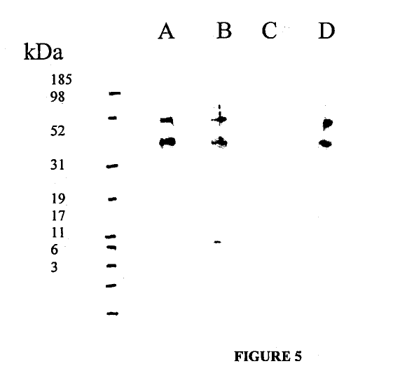

Avidin-HRP was used to visualize biotinylated (cell surface) proteins. Two major proteins at molecular weights of ^{˜}75 kDa and ^{˜}95 kDa were isolated that contained biotin label (FIG. 5). These two protein's binding to the S-tag K5 column could also be competed with excess rK5 or the K5 active site peptide (PRKLYDY) (FIG. 5, lane B and lane C) whereas the N-terminal peptide of K5 did not inhibit either protein from binding to the S-tag K5 column.

In particular, surface proteins on 1×10⁶ EAHY cells were labeled with NHS-biotin. The cells were washed 3× with PBS and lysed with M-pur. Cell lysates were mixed with (A) 100 nM S-tag-K5, (B) 100 nM S-tag-K5 plus 1 μM PRKLYDY, (C) 100 nM S-tag-K5 plus 10 μM cold rK5 and (D) 100 nM S-tag-K5 and N-terminal K5 peptide at 1 μM for 1 hour at room temperature. The K5 binding proteins were precipitated with S-protein-agarose. Bound proteins were eluted with 50 mM glycine buffer at pH 3.0 and run for PAGE analysis. Surface proteins (biotinylated) that bind K5 were visualized with avidin-HPR and a chemiluminescent substrate.

These results strongly suggest that GRP78 is found on the surface of stimulated endothelial cells and K5 binds to GRP78.

### EXAMPLE IV

### Visualization of GRP78 with a Chemiluminescent Substrate

Since the binding of rK5, to stimulated endothelial cells, is upregulated about 4-10 fold, as compared to starved cells, the level of GRP78 on endothelial cells should also be up regulated.

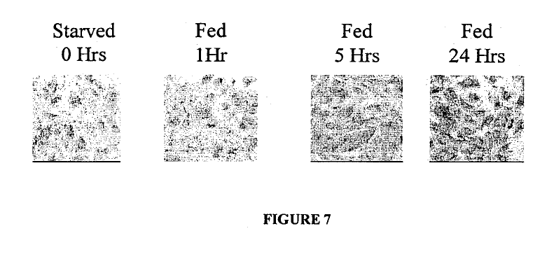

In FIG. 7, the levels of GRP78 protein were analyzed after cells were starved overnight and then fed complete media containing 100 ng/mp VEGF. At times indicated after feeding (see FIG. 7), whole cell lysates were run on PAGE and blotted with a polyclonal GRP-78-HRP antibody. The GRP78 was visualized with a chemiluminescent substrate.

Within 4 hours, the cellular protein levels of GRP78 dramatically increase after VEGF stimulation. This is also observed on endothelial cell surfaces by comparison of anti-GRP78 binding on starved as well as VEGF stimulated cells (FIG. 7). HMVEC cells, grown on glass slides, were starved for 26 hours, and then the media was replaced at various times with complete media containing 100 ng/ml VEGF. The cells were washed, fixed and stained for GRP78 with a goat polyclonal GRP78 antibody and an anti-goat HPR antibody. Determination of the amount of GRP78 present was determined by precipitation of a MTB substrate to give a dark brown color.

### EXAMPLE V

### Direct Binding of Recombinant Kringle 5 With GRP78

Ultra centrifugation with 50,000 MW cut off filters was used with iodinated recombinant dog K5 (I¹²⁵rK5(dog)) and GRP78 (bovine brain). The GRP78 and I¹²⁵rK5(dog) were incubated for 1 hour at room temperature. The solution was then transferred to ultracentrifuges and spun at 10,000×g for 2 min. The top chamber contained GRP78 (mw 78,000) and I¹²⁵rK5 (dog) that bound. The top and bottom solutions were counted for 1125. Column A: 1 nM I¹²⁵rK5(dog). Column B: 1 nM I¹²⁵rK5 (dog)+1.5 nM GRP78. Column C: 1 nM I¹²⁵rK5(dog)+1.5 nM GRP78+100 nM N-terminal K5 peptide, LLPDVETPSEED. Column D: 1 nM I¹²⁵rK5(dog)+1.5 nM GRP78+100 nM active site K5 peptide PRKLYDY. Column E: 1 nM I¹²⁵rK5(dog)+1.5 nM GRP78+1 nM rK5 (unlabeled). The GRP78 bound to the labeled rK5 causing its retention on the top of the filter. This binding can be inhibited by rK5 or the K5 active site peptide but not an inactive K5 N-terminal peptide.

1. A method of identifying a composition which inhibits activation of an endothelial cell receptor comprising the steps of:
a) constructing a vector comprising a nucleotide sequence encoding said endothelial cell receptor and a nucleotide sequence encoding a reporter molecule, said nucleotide sequence encoding said reporter molecule being operably linked to said nucleotide sequence encoding said endothelial cell receptor;
b) introducing said vector into a host cell for a time and under conditions suitable for expression of said endothelial cell receptor;
c) exposing said host cell to a composition which may inhibit activation of said endothelial cell receptor and a substrate specific for said reporter molecule; and
d) measuring the signal generated by reaction of said reporter molecule and said substrate in comparison to that produced by a control host cell, a smaller signal by said host cell of (c) indicating that said composition will inhibit activation of said endothelial cell receptor.

2. The method of claim 1, wherein said endothelial cell receptor is GRP78.

3. The method of claim 2, wherein said composition is kringle 5 (K5).

4. A method of identifying a composition which inhibits expression of an endothelial cell receptor comprising the steps of:
a) adding an antibody selected from the group consisting of a monoclonal antibody and a polyclonal antibody produced against said endothelial cell receptor to a solid phase;
b) adding known concentrations of said endothelial cell receptor, exposed to said composition, to said solid phase, in order to form a first complex between said antibody and said known concentrations of said endothelial cell receptor;
c) adding a second antibody to said first complex, selected from the group consisting of a monoclonal antibody and a polyclonal antibody produced against said endothelial cell receptor for a time and under conditions sufficient for formation of a second complex between said first complex and said second antibody;
d) contacting said second complex with an indicator reagent which comprises a signal-generating compound attached to an antibody against said antibody of said second complex, for a time and under conditions sufficient for formation of a third complex; and
e) detecting the presence of a measurable signal, absence of said signal indicating said composition inhibits expression of said endothelial cell receptor and presence of said signal indicating said composition does not inhibit expression of said endothelial cell receptor.

5. The method of claim 4 wherein said endothelial cell receptor is GRP78.

6. The method of claim 5 wherein a composition which inhibits expression of said endothelial cell receptor is K5.

7. A method of identifying a composition which binds to the GRP78 receptor comprising the steps of:
a) exposing said receptor to said composition for a time and under conditions sufficient for formation of a complex; and
b) determining presence or absence of said complex, presence of said complex indicating a composition which binds to said receptor.

8. The method of claim 7 wherein said compound is attached to an indicator molecule capable of generating a detectable signal.

9. The method of claim 7 wherein said compound which binds to said GRP78 receptor is K7 or a functional equivalent thereof.

10. A method of preventing or treating angiogenesis in a patient in need of said prevention or treatment comprising the step of administering to said patient in an amount of a composition which binds to at least one endothelial cell receptor sufficient to effect said prevention or treatment.

11. The method of claim 10 wherein said endothelial cell receptor is GRP78.

12. The method of claim 10 wherein said composition is K5.
